(19) United States
(12) Patent Application Publication
(10) Pub. No.: US 2008/0108790 A1
(43) Pub. Date: May 8, 2008

(54) S-Alkyl-Sulphenyl Protection Groups in Solid-Phase Synthesis

(75) Inventors: Stephane Varray, Sierre (CH); Oleg Werbitzky, Veyras (CH); Thomas Zeiter, Visperterminen (CH)

Correspondence Address: NIXON & VANDERHYE, PC, 901 NORTH GLEBE ROAD, 11TH FLOOR, ARLINGTON, VA, 22203, US

(73) Assignee: LONZA AG, BASEL, SWITZERLAND (CH)

(21) Appl. No.: 11/666,171

(22) PCT Filed: Oct. 26, 2005
(86) PCT No.: PCT/EP05/11476

§ 371 Date: Nov. 30, 2007

(30) Foreign Application Priority Data
Oct. 16, 2004 (EP) 04025395.7

(51) Int. Cl. C07K 1/06; C07K 7/06

(52) U.S. Cl. 530/328; 530/330; 530/334

(57) ABSTRACT

A novel method for on-resin formation of disulfide-borne cyclization of peptides is devised.

The present invention relates to a method of on-resin disulfide-bond formation in solid phase peptide synthesis (SPPS), and to respective peptide solid-phase conjugates.

A large variety of protection groups can be employed for protection of cysteine residues, e.g. trityl, acetamidomethyl-, t-butyl, trimethylacetamidomethyl, 2,4,6-triimethoxybenzyl, methoxytrityl, t-butylsulphenyl.

Most commonly, the trityl group is employed for simple protection during peptide synthesis. For protection of cysteines that are subsequently subjected to cyclization by means of cystine formation, acetamidomethyl (acm)-protection group along with iodine oxidation has been most widely employed (Kamber et al., 1980, Helv. Chim. Acta 63, 899-915; Rietman et al., 1994, Int. J. Peptide Protein Res. 44, 199-206). As a disadvantage, the spectrum of side-product impurities is substantially enhanced by using iodine, oxidizing susceptible side moities chain elsewhere, too. E.g. Tyr, Met may suffer from using iodine More importantly, oxidation with iodine may set free HI, the acid then eventually promoting deprotection of side chains and/or, most importantly, cleavage from resin. Therefore the method must be applied as a late finishing step in synthesis only, after cleavage from resin, if used at all.

The prior art knows a multitude of oxidating agents, beside iodine, which are added for allowing of cystine formation (examples derived from Albericio et al., in: Chan and White, eds., ‘FMOC Solid-phase Peptide Synthesis’, Oxford university Press 2000, p. 91 to 114: glutathione in aequeous buffer, DMSO, potassium ferricyanide, Ellman's reagent, 5,5′-dithiobis(2-nitrobenzoic acid), iodine, thallium (III)trifluoroacetate, alkyltrichlorosilane-sulphoxide, silver trifluoromethanesulphonate-DMSO mediated oxidation in strongly acidic medium.

Usually, all those methods give rise to undesireable, multiple side-products, require extended reaction times in the range of 10-20 hours for optimum yield and hence give ample opportunity to undesireable side-reactions. Volkmer-Engert et al. (Surface-assisted catalysis of intramolecular disulfide bond formation in peptides , J. Peptide Res. 51, 1998, 365-369) describe charcoal-catalyzed oxidative formation of disulfide bonds in water by using oxygen dissolved in the solvent, i.e. water. Careful controls showed that the pool of oxygen physically dissolved in the aequeous medium was necessary and sufficient to load the charcoal with oxygen for oxidation. Use of charcoal, as compared to traditional air-sparging in the absence of catalyst, accelerated the reaction rate dramatically.

The use of charcoal inevitably requires to carry out such reaction in homogenous solution but not on-resin; subsequent reaction steps of deprotection would not tolerate the continued presence of charcoal which is impossible to remove from the peptide-resin solid phase though. Cyclization accordingly takes place after cleavage from the resin, that is in solution. Cleavage from the solid support and global deprotection prior to cyclization is mandatory in this scheme. As a further disadvantage, Atherton et al. (1985, J. Chem. Perkin Trans. I., 2065) reported that the use of the popular both scavenger and acidolysis promoter thioanisol in acidic deprotection also resulted in partial, premature deprotection of acm, tert-butyl and tert-butylsulphenyl protected cysteines.

U.S. Pat. No. 6,476,186 devises intramolecular disulfide bonding of an octapeptide in acetonitril/water (1:1) in the presence of trace amounts of charcoal. The peptide was synthesized on 2-chlorotrityl resin and comprises apart from hydrophobic residues and the cysteines, a lysine and a threonine. Cysteines were protected with acid-labile trityl groups. Charcoal catalyzed cyclization took place after cleavage and deprotection in the aequeous solvent mixture.

It is an object of the present invention to devise a more simple and straightforward, other or improved method for synthesizing disulfide-bonded cyclic peptides by means of solid phase synthesis. This object is solved, according to the present invention, by a method of peptide synthesis comprising the steps of
a. synthesizing a peptide linked to a solid phase which peptide comprises at least two residues of a cysteine or a homo-cysteine, which cysteines are protected in their side chain each by a S-alkyl-sulphenyl protection group, wherein the alkyl may be further substituted with aryl, aryloxy, alkoxy, halogenated variants thereof or halogeno, and wherein the two protection groups may be the same or different, preferably they are protected in their side chain each by a S-tert.butyl-sulphenyl group, and
b. further reacting the peptide with a S-tert.Butyl-sulphenyl-protection group removing reagent, preferably reacting the peptide with a tertiary phosphine, and
c. cyclizing the peptide by means of disulfide bond formation in the presence of air and/or oxygen but, preferably, in the absence of a heterogenous catalyst.

The peptide according to the present invention may be any peptide comprising natural or non-natural amino acids such as e.g. homocysteines which homocysteines are preferably comprising 2-15 methylene groups and one thiol group in their side chains, homoarginine, D-cyclohexyl-alanine, ε-lysine, γ-lysine, Penicillinamide (Pen) or ornithine (Orn) or D-analogues of the natural L-amino acids. Preferably, the peptide comprises only natural amino acids or the D-analogues or the homo- or nor-anlogues thereof. The terms peptide backbone or main chain, side chain and the prefixes ‘nor-’ ‘homo-’ are construed in the present context in accordance the IUPAC-IUB definitions (Joint IUPAC-IUB Commission on Biochemical Nomenclature, ‘Nomenclature and symbolism for amino acids and Peptides’, Pure Appl. Chem., 56, 595-624 (1984). In its more narrow and preferred meaning, ‘homo-’ and ‘nor-’ amount to just one extra or missing, respectively methylen bridging group in the side chain portion, preferably with the exception of homocysteines which may be defined preferably as said above.

Particular attention must be paid to further side-chain protection of the amino acids forming the peptidic sequence, in particular when referring to further cysteine, homo- or nor-cysteine residues comprised in the peptide sequence that are intented to remain protected during rather than to participate in the cyclization reaction. Preferably, such further sulfhydryl-moiety comprising residues are protected by trialkylphosphine non-sensitive-, more preferably by tri-n-butylphosphine insensitive, protection groups, more preferably, such non-sensitive sulfhydrylprotection group is selected from the group comprising trityl-, tert.butyl-, acetamidomethyl-, alkylated acetamidomethyl-, alkylated trityl-protection groups.

On the more general level, side chain protection groups as commonly employed in the art (see e.g. Bodansky, M. , Principles of Peptide Synthesis, 2^{nd} ed. Springer Verlag Berlin/Heidelberg, 1993) may be used to protect susceptible side chains which could otherwise be modified in the coupling and deprotection cycles. Examples of amino acids with susceptible side chains are Cys, Asp, Glu, Ser, Arg, Homo-Arg, Tyr, Thr, Lys, Orn, Pen, Trp, Asn and Gln. Alternatively, a post solid-phase synthesis chemical modification of the peptide amide may be carried out to yield a desired side chain. For instance, as set forth amply in different references (EP-301 850; Yajima et al., 1978, J. Chem. Cos. Chem. Commun., p. 482; Nishimura et al., 1976, Chem. Pharm. Bull. 24:1568) homoarginine (Har) can be prepared by guanidation of a lysine residue comprised in the peptide chain or an arginine can be prepared by guanidation of an ornithine residue comprised in the peptide chain. This may be a less viable option though in view of the additional reaction steps required. Notably, coupling e.g. of Har requires extended coupling times and replenishing of coupling reagents. According to the present invention, it is one preferred embodiment to couple Arg or Har, preferably when being used as FMOC-Arg and FMOC-Har respectively, without the use of side chain protecting groups. This may be achieved by ensuring that post-coupling of the individual Arg or Har residue, the guanidino moiety is quantitatively protonated prior to any further coupling reactions and forms stable ion pair with the proton donor in organic solvent. This is preferably achieved by treating the resin bound peptide amide with an excess of the acidic coupling auxilliary BtOH or the like as described in more detail below in the experimental section. Another example of scavenging the charge of the guanidinium group is to use tetraphenyl borate salts of Fmoc-protected HAR for synthesis as set forth in U.S. Pat. No. 4,954,616.

The solid phase support or resin may be any support known in the art that is suitable for use in solid-phase synthesis. This definition of solid phase comprises that the peptide is bonded or linked via a functional linker or handle group to the solid phase or resin. Preferably the solid support is based on a polystyrene or polydimethylacrylamide polymer, as is customary in the art. According to the present invention, the peptide may be bonded via a suitable amino acid side chain, including e.g. the thiol moiety of a further cysteine residue of the peptide intended not to participate in the cyclization reaction, or may be bonded via the C-terminal α-carboxy group to a resin by means of e.g. an ether, thioether, ester, thioester or amide bond. Examples are solid supports comprising handle groups such as e.g. trityl, 2-chloro-trityl-, 4-methoxytrityl-, ‘Rink amide’ 4-(2′,4′-dimethoxybenzyl-aminomethyl)-phenoxy-, Sieber resin (9-amino-6-phenylmethoxy-xanthen-), 4-hydroxymethylphenoxyacteyl-, 4-hydroxymethylbenzoic acid (the latter requiring attachment of the first amino acid by means of p-dimethylaminopyridine-catalysed esterification protocol than can result in racemisation of susceptible amino acids, e.g. Trp and in particular cysteine, see Atherton, E. et al., 1981, J. Chem. Soc. Chem. Commun., p. 336 ff). Methods of providing thioester linkages to a resin are disclosed in detail and are farther referenced in WO 04/050686. Said reference also describes that thioester bonds are highly vulnerable to standard deprotection conditions used e.g. in Fmoc synthesis, and how use of a substitute base may overcome this problem. However, in a preferred embodiment of the present invention, thioester linkages for bonding of the peptide moiety to the solid-phase, be it in a C-terminal or side chain born linkage, are specifically disclaimed since subject to transthioesterification side reaction under at least slightly basic pH. Thioester linkages are vulnerable to treatment with S-tert.butyl-sulphenyl protection group removing agents, in particular those of the thiol reducing type such as β-mercapto-ethanol in near-stochiometric amounts or beyond. But also with tertiary phosphines this may happen, setting free cysteinyl-, homo-cysteinyl, or generally residues with free thiol groups the latter which allowing further of intramolecular transthioesterification reaction with a solid-phase-anchoring thioester bond. However, the intramolecular reaction may be strongly modulated by aspects of spacial distance and sequence dependent, conformational restraints and hence applying the above disclaimer is dependent both on the type of S-tert.butyl-sulphenyl-group removing agent and the specific sequence of a given peptide. Preferably and optionally, where thioester linkages for bonding of the peptide moiety to the solid-phase are employed, the S-tert.butyl-sulphenyl protection group removing agent is a phosphine, more preferably a tris-(C1-C8) alkyl-phosphine wherein the alkyl may be, independently, further substituted with halogeno or (C1-4)alkoxy or (C1-C4)ester. More preferably, the removing agent is a tris-(C2-C5)alkyl-phosphine wherein the alkyl may be further substituted, independently, with (C1-C2)alkoxy.

Notably, according to the present invention, S—S-bond-comprising resin handles such as the HPDI bifunctional hydroxy and disulfide handle described in Brugidou, J. et al., Peptide Research (1994) 7:40-7 and Mery, J. et al., Int. J. Peptide and Protein Research (1993), 42: 44-52) are of course excluded from the scope of the present invention since not allowing of on-resin cyclization.

On-resin cyclization according to the present invention allows of avoiding the problems arising from intermolecular side reaction and the dilution techniques or catalyst-surface absorption techniques usually employed for this reason.

Rink amide, Sieber resin (Tetrahedron Lett. 1987, 28, 2107-2110) or similar 9-amino-xanthenyl-type resins, PAL resins (Albericio et al., 1987, Int. J. Pept. Protein Research 30, 206-216) or the specially substituted trityl-amine derivatives according to Meisenbach et al., 1997, Chem. Letters , p. 1265 f.) are examples of linkage groups of a solid phase from which a Cα-carboxamid is generated or liberated upon cleavage of the peptide from the resin. In this sense solid phases giving rise to a carboxamid upon cleavage from resin, be it the carboxamid of a formerly acidic side chain or the C-terminus of the peptide, are termed amide-producing solid phases in the present context.

Preferably, the peptide is anchored to the solid phase by either an amide or ester bond via the C-terminus. More preferably, the solid phase is an acid-sensitive or acid-labile solid phase, even more preferably, it is an amide generating acid-labile solid-phase. Such acid-labile solid phases require at least 0.1% trifluoroacetic acid (TFA), more preferably at least 0.5% TFA in a polar aprotic solvent for cleavage from resin. Most preferably, the solid-phase is an acid-sensitive solid phase that is cleaved under weakly acidic conditions, that is 0.1 to 10% TFA in said solvent are sufficient to effect at least 90% cleavage efficiency upon incubation at room temperature up to 5 hours. Such highly acid-labile solid phase are e.g. 2-chlorotrityl resins, 4,4′-dimethoxytrityl resin, the related, trityl-based phenylalcohol resin such as e.g. Novasyn™ TGT derived from an conventional aminomethyl resin by acylation with Bayer's 4-carboxytrityl linker or a 4-methoxyphenyl, 4,4′-dimethoxyphenyl or 4-methyl-derivative of said linker, further Sieber resin, Rink amide resin or 4-(4-hydroxymethyl-3-methoxyphenoxy)-butyric acid (HMPB) resin, (4-methoxybenzhydryl-) or (4-methylbenzhydryl)-phenyl resins, the former said Sieber and Rink resin specifically giving rise to C-terminally amidated peptide upon acidolysis. Such acid-labile solid phases are particularly vulnerable to on-resin deprotection chemistries for side-chain protection groups and hence particular attention must be paid in these cases.

In case of side chain anchoring via C-terminal cysteine residue to the handle group of a solid support, the linking bond must be a thioether or thioester bond. Further suitable residues for side-chain anchoring are carboxy groups of acidic side chains, hydroxy groups and in particular the ε-amino group of lysine. It goes without saying that in case of side chain anchoring, that the C-terminal free carboxy group is generally to be protected by esterification or amidation prior to carrying out the first coupling reaction, e.g. by using FMOC-Lys-carboxamid for linking reaction of the side chain amino function to the solid phase.

In a preferred embodiment, one S-alkyl-sulphenyl-protected cysteine, preferably one S-tert.butyl-sulphenyl protected cysteine is the C-terminal residue of the peptide and is bonded via the carboxy-terminus by means of an ester or amide bond to the solid phase, with the proviso, that said linking bond is not a benzylester moiety but preferably is an acid-labile resin that is cleaved under weakly acidic reaction conditions as defined above. A C-terminal cysteine is particularly prone to subject to racemisation in acidic conditions, e.g. upon cleavage and/or deprotection under strongly acidic condition.

Eventually disclaimed heterogenous catalysts for air-borne, oxidative cyclization are e.g. charcoal, which is incompatible with use on a solid-phase. It may not be efficiently removed. Preferably, it relates to the absence of a catalytically effective or substantial amount of such heterogenous catalyst. Not using inappropriate catalyst when not required for the purposes of the present invention is a self-evident measure to the skilled artisan, though.

Coupling reagents for peptide synthesis are well-known in the art (see Bodansky, M., Principles of Peptide Synthesis, 2^{nd} ed. Springer Verlag Berlin/Heidelberg, 1993; esp. cf. discussion of role of coupling additives auxiliaries therein). Coupling reagents may be mixed anhydrides (e.g. T3P: propane phosphonic acid anhydride) or other acylating agents such as activated esters or acid halogenides (e.g. ICBF, isobutyl-chloroformiate), or they may be carbodiimides (e.g. 1-ethyl-3-(3-dimethylaminopropyl)-carbodiimide), activated benzotriazin-derivatives (DEPBT: 3-(diethoxyphosphoryloxy)-1,2,3-benzotriazin-4(3H)-one) or uronium or phosphonium salt derivatives of benzotriazol.

In view of best yield, short reaction time and protection against racemization during changing elongation, more preferred is that the coupling reagent is selected from the group consisting of uronium salts and phosphonium salts of the benzotriazol capable of activating a free carboxylic acid function along with that the reaction is carried out in the presence of a base. Suitable and likewise preferred examples of such uronium or phosphonium coupling salts are e.g. HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazol-1-yl-oxy-tripylolidinophosphonium hexafluorophosphate), PyAOP, HCTU (O-(1H-6-chloro-benzotriazole-1-yl)-1,1,3,3-tetramethyluronium hexafluorophosphate), TCTU (O-1 H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol- 1-yl)- 1,1,3,3-tetramethyluronium tetrafluoroborate), TOTU (O-[cyano(ethoxycarbonyl)methyleneamino]-N,N,N′,N′-tetramethyluronium tetrafluoroborate), HA-yU (O-(benzotriazol-1-yl)oxybis-(pyrrolidino)-uronium hexafluorophosphate.

Preferably, when using DEPBT or the like, uronium or phosphonium salt reagents, a further or second weak base reagent is needed for carrying out the coupling step. This is matched by base whose conjugated acid has a pKa value of from pKa 7.5 to 15, more preferably of from pKa 7.5 to 10, with the exclusion of an α-amino function of a peptide or amino acid or amino acid derivative, and which base preferably is a tertiary, sterically hindered amine. Examples of such and further preferred are Hünig-base ( N,N-diisopropylethylamine), N,N′-dialkylaniline, 2,4,6-trialkylpyridine or N-allyl-morpholine with the alkyl being straight or branched C1-C4 alkyl, more preferably it is N-methylmorpholine or collidine (2,4,6-trimethylpyridine), most preferably it is collidine.

The use of coupling additives, in particular of coupling additives of the benzotriazol type, is also known (see Bodansky, supra). Their use is particularly preferred when using the highly activating, afore said uronium or phosphonium salt coupling reagents. Hence it is further preferred that the coupling reagent additive is a nucleophilic hydroxy compound capable of forming activated esters, more preferably having an acidic, nucleophilic N-hydroxy function wherein N is imide or is N-acyl or N-aryl substituted triazeno, most preferably the coupling additive is a N-hydroxy-benzotriazol derivative (or 1-hydroxy-benzotriazol derivative) or is an N-hydroxy-benzotriazine derivative. Such coupling additive N-hydroxy compounds have been described in large and wide in WO 94/07910 and EP-410 182 and whose respective disclosure is incorporated by reference hereto. Examples are e.g. N-hydroxy-succinimide, N-hydroxy-3,4-dihydro-4-oxo-1,2,3-benzotriazine (HOOBt), 1-hydroxy-7-azabenzotriazole (HOAt) and N-hydroxy-benzotriazole (HOBt). N-hydroxy-benzotriazine derivatives are particularly preferred, in a most preferred embodiment, the coupling reagent additive is hydroxy-3,4-dihydro-4-oxo-1,2,3-benzotriazine. Ammonium salt compounds of coupling additives are known and their use in coupling chemistry has been described, for instance in U.S. Pat. No. 4,806,641.

In a further particularly preferred embodiment, the uronium or phosphonium salt coupling reagent is an uronium salt reagent and preferably is HCTU, TCTU or HBTU and even more preferably is used in the reaction in combination with N-hydroxy-3,4-dihydro-4-oxo-1,2,3-benzotriazine or a salt thereof. This embodiment is mainly preferred for use in chain elongation step of peptide synthesis after removal of the base-labile Nα-protection group, but may as well be used for lactamization reaction during side-chain cyclization.

In the context of the present invention, it is to be noted that HCTU and TCTU are defined as to be encompassed by the term ‘uronium salt reagent’ despite that these compounds and possible analogues have been shown to comprise an isonitroso moiety rather than an uronium moiety by means of crystal structure analysis (O. Marder, Y. Shvo, and F. Albericio “HCTU and TCTU: New Coupling Reagents: Development and Industrial Applications”, Poster, Presentation Gordon Conference February 2002), an N-amidino substituent on the heterocyclic core giving rise to a guanidium structure instead. In the present context, such class of compounds is termed ‘guanidinium-type subclass’ of uronium salt reagents according to the present invention.

In a further particularly preferred embodiment, the coupling reagent is a phosphonium salt of the benzotriazol such as e.g. BOP, PyBOP or PyAOP.

Deprotection of the base labile Nα may be carried out as routinely done in the art, e.g. with 20% piperidine in N-methyl morpholine when using standard Fmoc chemistry. Most widely, Fmoc or Boc protection chemistry for the N-terminus is routinely applied in solid phase synthesis but further optional Nα protection chemistries are known in the art and can be applied where not interfering with the present invention, that is to devise disulfide-borne peptide cyclization of the resin-conjugated peptide.

The S-alkyl-sulphenyl protecting groups protecting thiol groups of cysteine or homocysteine residues, as is shown in formula II, are removed according to the present invention by a reagent that typically is capable of removing, preferably substantially removing, the S-tert.butyl-sulphenyl-protection group from such residue. Removal of S-tert.butyl-sulphenyl protection groups from e.g. cysteine accomplished by means of reaction with tertiary phosphines has been described, for instance by using tributylphosphine (Atherton et al., 1985, J. Chem. Soc., Perkin I. 2057) and triethylphosphine (Huang et al, 1997, Int. J. Pept. Protein Res. 48, 290). The tert-butylsulphenyl group is also cleaved in an orthogonal fashion by means of thiol reagents such as e.g. β-mercapto-ethanol or dithio-threitol (DTT) as an option to using tertiary phosphines (Huang et al.,1997 Int. J. Pept. Protein Res. 48, 290; Rietmann et al., 1985, Reel. Trav. Chim. Pays-Bas, 1141). Preferably, the tertiary phosphine is triphenylphospine or is an (C1-C4) alkylated or (C1-C4)allcoxylated triphenylphosphine, such as e.g. tri-(p-methoxyphenyl)-phosphine or even more preferably is a trialkylphosphine wherein the alkyl may be the same or different, and wherein each alkyl is a C1 to C7 alkyl, preferably C1 to C4 allkyl, and may be branched or linear alkyl. Preferably, the alkyl is linear. Examples are methyl, ethyl, propyl, i-propyl, n-butyl, i-butyl. Tri-n-butyl-phosphine and tri-ethylphosphine are particularly preferred. The alkyl may be optionally further substituted with halogeno, (C1-C4)alkoxy such as e.g. and preferably methoxy ox ethoxy, or may be further substituted where amenable with the solvent system, carboxy or is, preferably, unsubstituted. Surprisingly, in one preferred embodiment according to the present invention, it has unexpectedly been found that disulfide cleavage by means of phosphines may also be used with the acid-labile resins cleavable in weakly acidic reaction conditions such as Sieber or 2-chloro-trityl (CTC) resin, for instance. It is also often overlooked that thiol reagens reduce and hence cleave disulfides by forming disulfide products themselves. Preferably, such thiol reagent is selected from the group consisting of erythro-2,3-dihydroxy-1,4-butanedithiol (or named meso-1,4-Dithioerythritol or DTE for short), DL-threo-2,3-dihydroxy-1,4-butanedithiol (or named rac-1,4-Dithiothreitol or DTT for short), L-threo-2,3-dihydroxy-1,4-butanedithiol, D-threo-2,3-dihydroxy-1,4-butanedithiol and mixtures thereof. Mixtures may comprise DTE and DTT, either in its racemic form or as an optically active preparation of DTT. More preferably, the thiol reagent is DTT which means D-,L- or any racemic or non-racemic mixture thereof. -DTT and DTE are also known as Cleland's reagent and Cleland's other reagent, respectively (Cleland, W. , Biochemistry 3,480-482,1964). Whereas in case of DTT, intramolecular ring closure is strongly favored, making it a stronger disulfide reducing agent and notably preventing formation of stable intermolecular disulfide adducts with DTT, in case of β-mercaptoethanol, any intermolecular reaction product, by way of disulfide exchange reaction, is feasible. Further newly formed disulfides may undergo further exchange reaction. The use of thiol reagents, most oftenly simple thiol reagents such as 2-mercaptoethanol, apparently owes to the fear of side reactions such as e.g. leakage from resin when using strongly nucleophilic tertiary phosphine reagents. By using DTT and the like, the inherent disadvantages of mono-thiol reagents may be avoided.

Cyclization is carried out according to the present invention in the presence of a first weak base in a polar, aprotic organic solvent in the presence of air and/or oxygen but notably in the absence of a heterogenous, rate-accelerating catalyst. Still then, and without precedent, the cyclization step, due to the method of the present invention, is remarkably efficient and requires only about 0.5 to 2 hours reaction time, allowing of literally quantitative, complete conversion of educt to the desired product under very mild reaction conditions (ambient temperature typically, expedient temperature range being 10° C. to 80° C. though reflux temperature of solvent must be taken into account of course). Conversion is complete. This is an outstanding achievement and has not yet been achieved in disulfid-bonding driven cyclization of peptide, nor have such simple, mild and rapid cyclization reaction conditions been devised earlier. No tedious mixing and separation problems for a heterogenous catalyst arise ever. Still, the reaction rate completely parallels that of the catalyst-borne reaction of the prior art. Due to the straightforward course of reaction, formation of side products is almost entirely avoided.

Suitable polar, aprotic solvents are e.g. acetonitril, dimethylformamide, dichloromethane, N-methyl-pyrrolidone, tetrahydrofurane. In contrast to water, such solvent usually may not physically dissolve relevant amounts of oxygen to supply the oxidative formation of disulfide bonds as has been described for aequeous catalyst systems before.

Accordingly, the supply of air, air/oxygen or pure oxygen must be paid attention to. Air/oxygen may be supplied by thorough stirring, vortexing, special design of propellers used for stirring, gas sparging into the liquid. The gas may be air or pure oxygen or air enriched with oxygen which is vented or sparged into the reaction liquid. In one particularly preferred embodiment, large or surface areas of the bottom and/or walls of the reactor vessel are punctured as to allow of sparging gas into the liquid, under thorough stirring. More preferably, the reaction vessel comprises a fritted bottom or a fritted section of least 50% surface area of the total surface of the bottom, as to allow of simultaneous stirring and venting by upsurging, bubbling air vented into the reaction liquid through that bottom.

The first weak base reagent is a weak base whose conjugated acid has a pKa value of from pKa 7.5 to 15, more preferably of from pKa 8 to 10, preferably, it is a tertiary, sterically hindered amine. Examples of such and further preferred are Hünig-Base (N,N-diisopropylethylamine), N,N-dialkyl-aniline, 2,4,6-trialkylpyrididine or N-alkyl-morpholine with the alkly being straight or branched C1-C4 alkyl such as methyl, ethyl, n-propyl, i-propyl, n-butyl, most preferably it is N-methylmorpholine, collidine (2,4,6-trimethylpyridine) or Hünig-Base.

Preferably the prior removal of the disulfide-bonded protection group according to the present invention, naotably the removal of the S-tert.butyl-sulphenyl group is effected in the presence of a first weak base reagent, for avoiding any risk of leakage from the resin by minor acidolysis, that is at a pH of from 7.5 to 12, more preferably of from 8 to 11. Optionally, by using polar aprotic solvents such as THF or acteonitril that are freely miscible with water, basic salts such as e.g. sodium acetate in aequeous solution may be used for that purpose. This embodiment is particularly preferred when using tertiary phosphines for said disulfide group cleavage or removal step. By combining a suitable oxygen supply concomittant with such disulfide protection group removal, it may be possible in another embodiment of the present invention, e.g. when using polar, aprotic organic solvent along with oxygen supply in the presence of a tertiary amine and when using tertiary phosphine for deprotection that is inert to oxygen, to carry out both disulfide deprotection and cyclization not only in a one-pot reaction but even as a single reaction step.

Due to the fact that the present method allows of on-resin cyclization, it further does not require tedious and yield decreasing strong dilution of peptide for favoring intra over intermolecular cyclization as previously required in most methods described in the prior art.

The on-resin operation mode of the invention allows of quick and efficient intra-molecular cyclization only, giving no chance of dimerization at all.

In a further preferred embodiment, the peptide is the peptide of formula I or II. The term protection group is to be construed as being protection group for a given side chain functionality or specific side chain which protection group is compliant with being used in standard tert-butyloxycarbonyl (Boc) or 9-fluorenylmethoxycarbonyl (Fmoc) solid phase peptide synthesis. Such protection groups and the use of specific protection groups for specific side chain functionalities are well-known in the art (s. Chan et al., ed., supra; Bodansky et al. supra).

A side chain group R1(o) for instance it not to be construed in the way as to refer to a single type of optionally protected amino acid side chain; each residue R1(1), R1(2) . . . may be unique or may be the same as at least one other residue. The same applies of course to radicals R2(x), R3(q).

Given the multitude of possible substructure, it is to be noted that the peptide of formulas I and II may also comprise well-known peptide backbone modifications that are commonly employed in peptide synthesis: cyclic amino acids such as D- or L-Pro, intermittent non-peptide moieties linking two peptidyl segments and being e.g. hairpin or β-turn mimetics or in particular backbone-modified dipeptidyl segments used in synthesis e.g. for introducing amide protected Asp-Gly(Hmb) segments avoiding aspartimide formation (Packman et al. 1995, Tetrahedron Lett. 36, 7523) or peptidomimetic, non-amide bonded dimeric segments of amino acids analogs having a backbone segments such as —CO—CH2— or CH2—NH2- instead of a peptide bond (a review of useful peptidomimetic segments can be found e.g. in Morley, J., Trends Pharm. Sci. (1980), pp. 463-468).

Preferably, the two cysteines that are going to be disulfide-connected in cyclization are spaced apart by at least two amino acid residues (or the like). A spacing of i+3 is typical of an α-helical peptide conformation and allows of optimal, spacial juxtaposition for disulfide bonding. In this way, cyclization is facilitated. Below, the constraint exercised by the backbone in view of the possible, more stable conformations is rendering cyclization more difficult. However, it is to be noted that the incorporation of pseudo-prolines as helix breakers or of D-amino acids as inducers of beta-turn conformations in the spacer segment of the peptide moiety, that is as one of the amino acids encompassing a radical R2(x), is strongly modulating this simple rule, which is highly structure dependent accordingly.

In one further embodiment, it may be possible to synthesize a pep tide on a solid phase not by permanent, covalent attachment of the peptidyl moiety to a solid-phase but by non-covalent, reversible attachment to the solid-phase by means of a stable metal chelate complex (press release October/November 2004 made by Lonza A G, Basel, Switzerland jointly with AplaGen GmbH, Baesweiler, Germany, October 2004), similar to the hexa-His tag technology employed in protein purification since long. Such non-covalent solid-phase linkage or similar, future embodiments are encompassed by the present invention as well and the preferred modes of operating the present invention set forth above and in the claims below apply to this embodiment, substituting the aforementioned linkage or bonding to the resin or handle with the non-covalent bonding feature of said present embodiment.

A further object of the present invention are the respective, solid-phase borne peptides or solid-phase-peptide conjugates, respectively. The relevant definitions given above and below apply likewise to such object, alone or in combination.

Accordingly said further object of the present invention is a peptide of formula I or II,

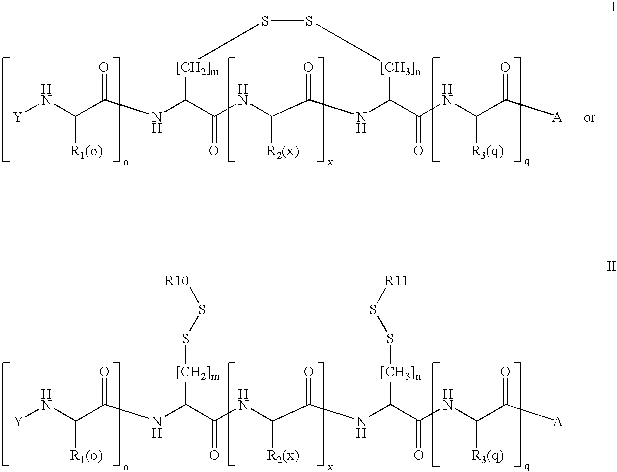

wherein m,n=1-15, preferably m,n=1 or 2, wherein Y═H or Y is first a protection group, preferably Y is an non-base labile protection group, o,x,q each separately is 0-200 and wherein R1(o), R2(x), R3(q) each, independently, is a side chain of an amino acid selected from the group consisting of natural amino acids including cyclic amino acids, non-natural amino acids including cyclic amino acids or non-amide bonded dipeptidyl segments, non-natural derivatives of natural amino acids or analogues thereof and wherein said amino acid chain may each farther comprise a second protection group that may be the same or different for individual side chains, and wherein A is a resin or resin handle or wherein optionally an individual R2(x) or R3(q) radical is linked to a resin or resin handle with the proviso that then A is selected from the group comprising OH, NH₂, NR′₁H or NR′₁R′₂ with R′_{1,2} being C1 to C4 alkyl, and wherein R10, R11 each are alkyl which may be further substituted with aryl, aryloxy, alkoxy, halogenated variants thereof or halogeno and may be the same or different.

Preferably, x=2-200. More preferably, o, x, q each separately is 1-100, preferably 2-50, or wherein x is 2-100, preferably x is 3-50. Again more preferably, q=0 and more preferably in this context further o is 0-50 and wherein x is 2-100, preferably wherein x is 2-50.

### EXPERIMENTS

The overall synthetic strategy is set forth in table I underneath:

TABLE I

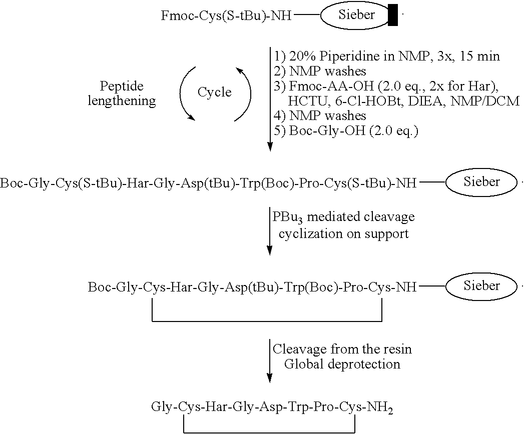

### 1.1 FMOC Solid Phase Synthesis of Linear Peptide Fmoc-Gly-Asn(tBu)-Trp(Boc)-Pro-Cys(S-tBu)-Sieber

Synthesis of FMOC-Cys(S-tBu)-OH has been described before (Rietman et al., 1994, Synth. Commun. 24, p. 1323 f). Sieber resin was a Novabiochem™ product of 100-200 mesh (US Bureau of Standards mesh sizing), the matrix material being divinylbenzene-crosslinked polystyrene, and was purchased from Calbiochem-Novabiochem (belonging to EMD Biosciences, California/U.S.A.). All FMOC amino acids, including FMOC-Cys(S-tBu)-OH (cat. No. B-1530) were purchased from Bachem AG (Bubendorf, Switzerland).

Loading of resin was at 0.52 mmol/g and of a total of 10 g Sieber resin. Coupling time for loading was twice the standard coupling time, namely 60 min. in total. Couplings were conducted with 2 eq. each of respective amino acid in the presence of 1 eq. each of 6-chloro-HOBt, TCTU, Hünig-Base (Disopropylethylamine), in dichloromethane. Washes were with N-methyl-pyrrolidone (NMP).

FMOC deprotection was done by 3 cycles of 15 min. 10% piperidine in N-methyl-pyrrolidone; efficiency of cleavage and completion of synthesis was analysed by Ninhydrin reaction and reverse phase HPLC, respectively.

### 1.2 Elongation of Peptide from 1.1 to Boc-Gly-Cys(S-tBu)-Har-Gly-Asp(tBu)-Trp(Boc)-Pro-Cys(S-tBu)-Sieber

The coupling of the FMOC-Har residue (Bachem, Burgendforf, Switzerland) took place in the presence of 1 eq. HOBt (for keeping the Guanidino group protonated) per eq. amino acid; the FMOC amino acid was preincubated with HOBt and diisopopyl-carbodiimid in NMP and was then mixed with the resin. Har coupling took 180 min. (other aa: 30 min.) followed by a second cycle with replenished reagents of about 60 min. In this way, standard 99.8% coupling efficiency as for the other residues could be matched. FMOC cleavage took place as before. Notably, after FMOC cleavage und subsequent NMP washes, repeated washing with HOBt was done to prevent further swelling of the resin

### 1.3 Deprotection of Protected (S-tBu)-Cysteines with Bu₃P

The resin product of step 1.2 was suspended and washed three times in tetrahydrofurane (THF). The reaction was carried out for 1 h at room temperature with 50 eq. tributylphosphine made up as 1 9%(v/v) PBu₃/77% (v/v) THF/4%(v/v) saturated aequeous solution of sodium acetate; precipitating salt was filtered off prior to use. Reaction proceeded uniformly to give one dominant product peak. The yield was determined by reverse phase HPLC and was found to amount to 98.9% correct product.

### 1-4 Cyclization to Yield Boc-Gly-Cyclo(Cys-Har-Gly-Asp(tBu)-Trp(Boc)-Pro-Cys)-Sieber

The swollen peptide-resin conjugate from exp. 1.3 was washed three times in NMP. Cyclization was done by incubating the resin for 1 h at room temperature with 6% DIEA (Hünig-Base) in NMP; reaction was carried out in a vertical glass vessel which comprised a horizontally bisecting, sealed-in G3 (16-40 μm) glass frit in its lower portion. The glass frit or fritted plate was vented with air from below, allowing of air bubbling across the entire cross-section of the solvent-covered reactant space above the frit in which the resin was floating by the bubbling air from underneath. A strictly pure, uniform product is obtained, no distinct or shattered side products do show off after this reaction step. The conversion to product was 100%, as determined independently by both reverse phase HPLC and LC-MS. RP-HPLC was carried out on a Hypersil-Keystone™ Betabasic (Thermo Electron Corp., Waltham Mass./U.S.A.) C18 150×4.6 mm column, with an injection volume of 15 μl and detection at 262 nm at a column temperature of 35° C. Gradient run is

| | |
|---|---|
| Time | Acetonitrile (0.1% TFA)/Water (0.1% TFA) |

| | | |
|---|---|---|
| 0 | 60 | 40 |
| 5 | 97 | 3 |
| 16 | 97 | 3 |
| 17 | 60 | 40 |

### 1.6 Global Deprotection

Global deprotection is prepared by swelling the resin three times in dichloromethane (DCM). Cleavage reaction phase mixture is prepared as to be made up from
86.5% TFA (785 eq.)
4.5% Thioanisol (36.5 eq.)
3% Phenol (32.4 eq.)
3% DCM (38 Eq.)
3% H2O (178 Eq.)

Reaction takes place at 15° C. for 2 h on an slowly rotating orbital shaking device. Reaction is terminated and product is precipitated, after filtering off the resin, by dropwise addition of tert.butyric acid methyl ester. The product is a uniform peak; no major side product can be detected.—the above conditions of global deprotection have been tested on a control and found not to affect preformed disulfide bridges in peptides.

### 2. Deprotection of Protected (S-tBu)-Cysteines with DTT

As an option to the deprotection step in 1.3, deprotection is carried out with DTT instead of phosphin essentially as described there. DTT is either rac- or L-DTT, obtainable from Biosynth AG/Switzerland. The resin product of step 1.2 was suspended and washed three times in dimethylformamide (DMF). 50 eq. of DTT were used, made up as DMF/DTT (1:1) and the reaction time was extended to 3-5 hours at room temperature. Subsequently, the peptide-resin was treated exactly as described in section 1.4-1.6 above. Yields obtained perfectly matched that of 1.6, with similar purity.

### 3. Cyclisation of Boc-D-Phe-Cys(S-tBu)-Tyr(tBu)-D-Try(Pbf)-Lys(Boc)-Val-Cys(S-tBu)-Trp(Pbf)-Sieber

Vapreotide, a Somatostatin peptidagonist, is synthesized essentially as described above in section 1.1. Further processing is carried out essentially as described in sections 1.2-1.6, providing the deprotected Vapreotide-carboxamide in excellent yield and purity. Optionally, deprotection according to section 2. is carried out, likewise with very good result.

### 4. Cyclisation of Boc-Lys(Boc)-Cys(S-tBu)-Asn-Thr(Trt)-Ala-Thr(Trt)-Cys(S-tBu)-Ala-Thr(Trt)-Gln-Arg(Pbf)-Leu-Ala-Asn-Phe-Leu-Val-His-Ser(Trt)-Ser(Trt)-Asn-Asn-Phe-Gly-Pro-Ile-Leu-Pro-Thr(Trt)-Asn-Val-Gly-Ser(Trt)-Asn-Thr(Trt)-Tyr-Sieber

Pramlintide peptide, a 37-mer, is synthesized and cyclized essentially as described above in sections 1.1-1.6. As compared to the yield of linear peptide, cyclization itself is quantitative. However, full length C to N-terminal linear synthesis give mediocre yield, due to several difficult individual coupling steps.

### 5. Cyclization of Fmoc-Cys(S-tBu)-Asn-Thr(Trt)-Ala-Thr(Trt)-Cys(S-tBu)-Ala-Thr(Trt)-Gln-Arg(Pbf)-Leu-Ala-Asn-Phe-Leu-Val-His-Ser-Trt)-Ser(Trt)-Asn-Asn-Phe-Gly-Pro-Ile-Leu-Pro-Pro -Thr(Trt)-Asn-Val-Gly-Ser(Trt)-Asn-Thr(Trt)-Tyr-Rink

Synthesis is essentially carried out as described in section 4. above, except that the last Lys residue is added after cyclization reaction in an additional coupling cycle, that synthesis is carried out on a Rink amide resin and that prior to global deprotection, cleavage is carried out under weakly or mildly acidic condition: Cleavage from resin is achieved with 3 cycles of 15 min. each at 15° C., 2% (w/w) TFA, 1% (w/w) triethylsilane (TES) in dichloromethane. The reaction is stirred by nitrogen bubbling. After each cycle, cleavage reaction is directly quenched by pouring the whole reaction broth into dilute pyridin (pyridine/ethanol 1:9 (v/v)). Resin is then removed by filtration with a frit. Filtrates are pooled and concentrated under vacuo (RotaVap), and washed with DCM.

### 6. Cyclization of Boc-Lys(Boc)-Cys(S-tBu)-Asn-Thr(Trt)-Ala-Thr(Trt)-Cys(S-tBu)-Ala-(ψ^{Me,Me}pro)Thr-2-CTC

Synthesis and cyclization is essentially carried out as described in sections 1.1-1.5 above, except that 2-chlorotrityl-polystyrene resin (CBL Patras, Greece) is used as a solid phase and that the DTT method according to section 2 is used instead of 1.3/phosphine method. Further, cleavage under mildly acid condition without side chain deprotection is used, essentially as described in section 5. Good yields are obtained. Fragment synthesis serves as an optional route to Pramlintide synthesis: the cylized, bridging-cystine comprising but still protected peptide is then subjected to conventional fragment coupling technique with a C-terminal, residual fragment of Pramlintide using standard peptide coupling chemistry with TCTU wherein the C-terminal, protected fragment is harbored either on solid-phase or, preferably, in liquid phase, too.

1. Method of peptide synthesis, comprising the steps of
a. synthesizing a peptide linked to a solid phase which peptide comprises at least two residues of a cysteine or a homo-cysteine, which cysteines are protected in their side chain each by a S-alkyl-sulphenyl protection group, wherein the alkyl may be further substituted with aryl, aryloxy, alkoxy, halogenated variants thereof or halogeno, and wherein the two protection groups may be the same or different and
b. reacting the peptide with a S-alkyl-sulphenyl-protection group removing reagent and
c. cyclizising the peptide on-resin by means of disulfide bond formation in the presence of air and/or oxygen which is sparged into the liquid an Al in the absence of a heterogenous, rate-accelerating catalyst, and
d. cleaving the peptide from the resin

2. Method according to claim 1, characterized in that said cysteines are spaced apart by at least 3 residues.

3. Method according to claim 1, characterized in that the solid phase resin is an acid-labile resin.

4. Method according to claim 1, characterized in that the peptide has at least one further side chain protection group which protection group is not a S-alkyl-sulphenyl protection group including differently protected further cysteine or homo-cysteine residues.

5. Method according to claim 1, characterized in that the homo-cysteine comprises
2-15 methylene groups and one thiol group in its side chain.

6. Method according to claim 1, characterized in that the removal of the S-alkyl-sulphenyl groups is accomplished by reacting the peptide with a trialkylphosphine or a thiol reagent.

7. Method according to claim 1, characterized in that the peptide is cyclized in the presence of a weak base in a polar, aprotic solvent and wherein the bottom and/or walls of the reactor vessel are punctured or fritted as to allow of sparging gas into the liquid, as to allow of venting air into the liquid.

8. Method according to claim 1, characterized in that the linkage of the peptide to the solid phase is acid labile, preferably labile in 60% TFA in dichloro-methane at room temperature.

9. Method according to claim 5, characterized in that the peptide comprises 2-100 amino acid residues.

10. Method according to claim 1, characterized in that the peptide is cleaved off from the resin under global deprotection.

11. Method according to claim 1, characterized in that the linkage of the peptidyl moiety to the solid phase resin is not a thioester or disulfide bond linkage.

12. Peptide of formula II, 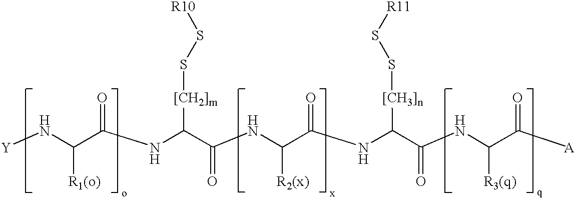
wherein m,n=1-15, preferably m,n=1 or 2, wherein Y═H or Y is first a protection group, o,x,q each separately is 0-200 and wherein R1(o), R2(x), R3(q) each, independently, is a side chain of an amino acid selected from the group consisting of natural amino acids including cyclic amino acids, non-natural amino acids including cyclic amino acids or non-amide bonded dipeptidyl segments, non-natural derivatives of natural amino acids or analogues thereof and wherein said amino acid chain may each further comprise a second protection group that may be the same or different for individual side chains, and
wherein A is a resin or resin handle or wherein optionally an individual R2(x) or R3(q) radical is linked to a resin or resin handle with the proviso that then A is selected from the group comprising OH, NH₂, NR′₁H or NR′₁R′₂ with R′_{1,2} being C1 to C4 alkyl, and wherein R10, R11 each are alkyl which may be further substituted with aryl, aryloxy, alkoxy, halogenated variants thereof or halogeno and may be the same or different.

13. Peptide according to claim 12, characterized in that x 2-200.

14. Peptide according to claim 12, characterized in that x is 2-100, preferably that x is 3-50.

15. Peptide according to claim 12, characterized in that q=0.

16. Peptide according to claim 15, characterized in that o is 0-50 and wherein x is 2-100, preferably wherein x is 2-50.
